(19) United States
(12) Patent Application Publication
(10) Pub. No.: US 2018/0268735 A1
(43) Pub. Date: Sep. 20, 2018

(54) MOBILE TERMINAL-BASED LIFE COACHING METHOD, MOBILE TERMINAL, AND COMPUTER-READABLE RECORDING MEDIUM, ONTO WHICH METHOD IS RECORDED

(71) Applicant: UNIVERSITY-INDUSTRY COOPERATION GROUP OF KYUNG HEE UNIVERSITY, Yongin-si, Gyeonngii-do (KR)

(72) Inventor: Chang-ho JIHN, Seongnam-si, Gyeonggi-do (KR)

(21) Appl. No.: 15/534,648

(22) PCT Filed: Dec. 9, 2015
(86) PCT No.: PCT/KR2015/013463

§ 371 Date: Jun. 9, 2017

(30) Foreign Application Priority Data
Dec. 9, 2014 (KR) 10-2014-0176278
Dec. 9, 2015 (KR) 10-2015-0174916

(51) Int. Cl. G09B 19/00; A63B 24/00; G09B 5/06; A61B 5/00; A61B 5/0205; A61B 5/16

(52) U.S. Cl. CPC G09B 19/00; G09B 19/0084; G09B 19/0092; A63B 24/0062; G09B 5/06; G16H 20/10; A61B 5/02055; A61B 5/165; A61B 5/486; A61B 5/6898; A61B 5/0002

(57) ABSTRACT

Provided are a mobile device-based life coaching method, a mobile device, and a computer-readable recording medium having recorded the method thereon. The mobile device-based life coaching method includes (a) receiving user type information using a user type information input program, (b) extracting standard guideline information corresponding to the user type information using a standard guideline information extraction program, (c) receiving sensed information from one or more sensing units using a sensing program, and (d) extracting condition and situation information of a user from the sensed information and outputting life coaching information corresponding to the standard guideline information based on the extracted condition and situation information at an appropriate time and place in an appropriate way using a life coaching program.

### TECHNICAL FIELD

The present invention relates to a mobile device-based life coaching method, a mobile device, and a computer-readable recording medium having recorded the method thereon and, more particularly, to a mobile device-based life coaching method, a mobile device, and a computer-readable recording medium having recorded the method thereon, by which a life coaching function helpful to a user may be provided using a mobile device capable of extracting a condition and situation of the user.

### BACKGROUND ART

Since many people do not pay much attention to healthcare due to their hectic routines, it is necessary to achieve health improvement by checking occasions for healthcare and continuously providing coaching services. However, as it says “three hour wait for three minutes with the doctor”, a doctor meets a large number of patients within a short time and thus doctors do not have sufficient time to give advices on real lives of patients.

Standard behaviors useful to individuals or groups may be required of groups of people, e.g., office workers, patients, and students.

For example, standard behaviors required of office workers, standard behaviors required of patients, and standard behaviors required of students are different and, statistically, the standard behaviors have similarity within a group.

That is, basic standard times for office workers, e.g., attendance time, lunch time, and quitting time, are mostly fixed, and occasions such as business trips or after-work parties may also be standardized. These standard behaviors may be similarly applied to patients or students.

Meanwhile, the Internet consists of a huge number of computers and computer networks for globally interconnecting the computers using communication links.

The interconnected computers exchange information using a variety of services, e.g., e-mails, Gopher, and World Wide Web (WWW). The World Wide Web allows a server computer system (e.g., a web server or website) to transmit graphical web pages of information to a remote client computer system.

The remote client computer system may display various web pages received from the server computer.

Due to rapid development of wireless communication networks including a mobile communication network such as a second generation (2G), third generation (3G), fourth generation (4G), fifth generation (5G), or long term evolution (LTE) network, Wi-Fi communication network, Bluetooth communication network, cellular communication network, code division multiple access (CDMA) communication network, Ethernet communication network, WiMAX communication network, local area network (LAN), wide area network (WAN), radio frequency (RF) communication network, infrared communication network, and optical communication network, a variety of systems using the wireless communication networks are being developed.

### DETAILED DESCRIPTION OF THE INVENTION

### Technical Problem

Existing smartphones, smartpads, smartwatches, or various wearable devices detect blood pressure, pulse, the amount of exercise, or the amount of activity of a user, and shows the detected result to the user. The user may set a normal range of the detected one-dimensional information and receive a warning or notification when out of range. However, if the normal range is set and fixed by a device manufacturer, conditions or situations of a certain user may not be considered. The normal range of the amount of exercise may be differently set depending on a health condition of the user, e.g., whether the user has a disease, and the normal range of pulse may be differently set depending on a situation of the user, e.g., participation in a sport game. The number of types of information to be monitored may differ depending on conditions and situations of the user and the information may be obtained from another device. As such, the manufacturer cannot set optimal normal ranges in consideration of conditions and situations of users and all types of information to be collected. This principle is equally applied to a case in which users directly input normal ranges.

The present invention provides a mobile device-based life coaching method, a mobile device, and a computer-readable recording medium having recorded the method thereon, by which, if user type information is input, various types of life coaching information useful to a user may be provided to the user in accordance with standard guideline information based on a condition and situation of the user, by which, if the user is a patient and diagnosis information and prescription information given by a doctor are input, for example, in the form of a photo, the user type information may be extracted from the input diagnosis information and the life coaching information corresponding to the user type information may be automatically output from a mobile device, e.g., a smartphone, of the patient based on the condition and situation of the user at an appropriate time and place in an appropriate way, by which the life coaching information stored in the mobile device, e.g., a smartphone, may be provided to the patient at an appropriate place and time in the form of notification/recommendation/warning information to allow the user to comply with the life coaching information, and by which coaching data per disease and compliance information may be stored and visualized to check non-compliance and to enable active self-care.

Using a method and system for determining possible conditions and situations based on the type of a user to be monitored, generating standard guideline information for inducing desirable behaviors of the user based on information collectable based on all combinations of the conditions and situations, continuously monitoring a condition and situation of the user, and providing coaching data to the user based on the condition and situation of the user and the standard guideline information, types of information to be monitored, information obtaining methods, information obtaining timings, behaviors to be induced, behavior inducing methods, etc.

may be automatically determined by merely proving user type information without setting normal ranges of all types of information based on the conditions and situations of the user and the number of types of information to be obtained. The user type information may be induced from another type of information. The user type information may be expressed as sequential codes such as “type A”, “type B”, or “type C”, or as meaningful codes such as “influenza-early stage-infant”. An example of the information from which the user type information is inducible includes “prescription information” given to a patient in an early stage of influenza.

However, the scope of the present invention is not limited to the above description.

### Technical Solution

According to an aspect of the present invention, there is provided a mobile device-based life coaching method using a mobile device carriable by a user, the mobile device including a user type information input program for receiving user type information, a standard guideline information extraction program for extracting standard guideline information corresponding to the user type information, a sensing program for receiving sensed information from one or more sensing units, a life coaching program for extracting condition and situation information of the user from the sensed information and outputting life coaching information corresponding to the standard guideline information based on the extracted condition and situation information at an appropriate time and place in an appropriate way, a user type information database for storing the user type information, a standard guideline information database for storing the standard guideline information, a sensed information database for storing the sensed information, and a life coaching information database for storing the life coaching information, the mobile device-based life coaching method including (a) receiving the user type information using the user type information input program, (b) extracting the standard guideline information corresponding to the user type information using the standard guideline information extraction program, (c) receiving the sensed information from the sensing units using the sensing program, and (d) extracting the condition and situation information of the user from the sensed information and outputting the life coaching information corresponding to the standard guideline information based on the extracted condition and situation information at an appropriate time and place in an appropriate way using the life coaching program.

Diagnosis information from which the user type information is extractable may include at least diagnosis information given by a medical expert and information about a group to which the user belongs, the mobile device may further include a diagnosis information input program for receiving the diagnosis information, and a diagnosis information database for storing the diagnosis information, and the mobile device may include at least one selected from the group consisting of a smartphone, a smartwatch, a wearable smart device, and combinations thereof.

The diagnosis information may include at least one selected from the group consisting of a diagnosis report photo file, text diagnosis file, diagnosis message file, email file, character recognition file, scan file, and electronic document file, which are storable in only the mobile device to protect personal information, and combinations thereof, and the diagnosis information input program may include a character recognition program for recognizing a diagnosed disease name or a prescribed medication name in characters included in the file.

The standard guideline information may include at least one selected from the group consisting of medication coaching information including medication time/medication compliance/over-the-counter medication information/side effects/prescription of multiple medications, hygiene coaching information including teeth brushing/hand washing/shower or bath/laundry, environment coaching information including temperature/humidity/fine dust/yellow dust/radioactivity, sanitation coaching information including mask wearing/gloves/sanitation/disinfection, exercise coaching information including exercise time/exercise intensity/the number of exercises/calorie consumption, health coaching information including mealtime/wake-up time/bedtime/habits/food tastes, and combinations thereof.

The life coaching information may include at least one selected from the group consisting of notification information indicating time/things to do, warning information indicating things not to do/things to be careful of, recommendation information indicating things recommended to do, and combinations thereof.

The sensing units may include at least one selected from the group consisting of a camera, a global positioning system (GPS) sensor for sensing a location of the user, a Wi-Fi sensor for sensing the location using Wi-Fi, an acceleration sensor for sensing an amount of exercise, an amount of activity, or a direction, a gyroscope sensor, a microphone sensor for sensing speech of the user/talks with other people/ambient noise/coughing sound/groaning sound, a speech recognizer, a motion sensor for sensing motion, a proximity sensor, an infrared sensor, an illumination sensor for sensing a sleep environment or lighting, a character recognizer for sensing a stress or depression index based on search words, a photo recognizer for recognizing a facial expression of the user, a temperature sensor for sensing temperature, a temperature recognizer for sensing temperature based on temperature data, a message recognizer for sensing psychology of the user based on messages, a Bluetooth sensor for sensing other people in proximity, an application sensor for sensing use of applications, a call volume sensor for sensing a call volume, a weather recognizer for receiving weather information from a weather center, a heart rate sensor, a blood pressure sensor, a body temperature sensor, a blood flow sensor, and combinations thereof.

The condition and situation information may include at least one selected from the group consisting of meal information for analyzing the condition and situation of a meal based on time/location/talks, going out information for analyzing the condition and situation of going out based on the location of the user who leaves or returns to a residence or office, sleep information for analyzing the condition and situation of sleeping and rising based on a mobile device manipulation time or intensity of light, exercise information for analyzing the condition and situation of exercise based on the location or acceleration of the user, emotion information for analyzing the condition and situation of an emotion based on a sensed talks/message/photo, eating information for analyzing the condition and situation of eating based on a sensed talks/message/location, after-work party information for analyzing the condition and situation of an after-work party based on Bluetooth/location/talks, environment information for analyzing the condition and situation of an environment based on temperature/location/time/body temperature, and combinations thereof.

The life coaching information may be output using at least a displayer, speaker, or vibrator of the mobile device in (d), the mobile device may further include a compliance information collection program for collecting compliance information of the user, and a compliance information database for storing the compliance information, and the mobile device-based life coaching method may further include (e) collecting the compliance information of the user using the compliance information collection program, after (d).

The mobile device may further include a visualization program for converting the condition and situation information or the compliance information into visualized information and outputting the visualized information, and a visualized information database for storing the visualized information, and the mobile device-based life coaching method may further include (f) converting the condition and situation information or the compliance information into the visualized information and outputting the visualized information using the visualization program, after (d).

The mobile device may further include a user information input program for receiving user information, and a user information database for storing the user information, and the mobile device-based life coaching method may further include (g) receiving the user information using the user information input program, before (a).

According to another aspect of the present invention, there is provided a mobile device capable of performing a mobile device-based life coaching method, the mobile device including a user type information input program for receiving user type information, a standard guideline information extraction program for extracting standard guideline information corresponding to the user type information, a sensing program for receiving sensed information from one or more sensing units, a life coaching program for extracting condition and situation information of the user from the sensed information and outputting life coaching information corresponding to the standard guideline information based on the extracted condition and situation information at an appropriate time and place in an appropriate way, a user type information database for storing the user type information, a standard guideline information database for storing the standard guideline information, a sensed information database for storing the sensed information, a life coaching information database for storing the life coaching information, and a controller programmed to receive the user type information using the user type information input program, to extract the standard guideline information corresponding to the user type information using the standard guideline information extraction program, to receive the sensed information from the sensing units using the sensing program, and to extract the condition and situation information of the user from the sensed information and output the life coaching information corresponding to the standard guideline information based on the extracted condition and situation information at an appropriate time and place in an appropriate way using the life coaching program.

According to another aspect of the present invention, there is provided a computer-readable recording medium having recorded thereon a mobile device-based life coaching method using a mobile device carriable by a user, the mobile device including a user type information input program for receiving user type information, a standard guideline information extraction program for extracting standard guideline information corresponding to the user type information, a sensing program for receiving sensed information from one or more sensing units, a life coaching program for extracting condition and situation information of the user from the sensed information and outputting life coaching information corresponding to the standard guideline information based on the extracted condition and situation information at an appropriate time and place in an appropriate way, a user type information database for storing the user type information, a standard guideline information database for storing the standard guideline information, a sensed information database for storing the sensed information, and a life coaching information database for storing the life coaching information, the mobile device-based life coaching method including (a) receiving the user type information using the user type information input program, (b) extracting the standard guideline information corresponding to the user type information using the standard guideline information extraction program, (c) receiving the sensed information from the sensing units using the sensing program, and (d) extracting the condition and situation information of the user from the sensed information and outputting the life coaching information corresponding to the standard guideline information based on the extracted condition and situation information at an appropriate time and place in an appropriate way using the life coaching program.

### Advantageous Effects

According to some embodiments of the present invention, various types of life coaching information useful to a user may be provided to the user based on a condition and situation of the user, the life coaching information may be automatically output from a mobile device, e.g., a smartphone, of a patient based on the condition and situation of the user at an appropriate time and place in an appropriate way, the life coaching information may be provided to the patient at an appropriate place and time in the form of notification/recommendation/warning information to allow the user to comply with the life coaching information, coaching data per disease and compliance information may be stored and visualized to check non-compliance and to enable active self-care, life coaching services for a disease may be continuously provided, social and economic burdens due to medical expenses may be reduced, and medical information industries may be vitalized. However, the scope of the present invention is not limited to the above effects.

### DESCRIPTION OF THE DRAWINGS

FIG. 1 is a block diagram of a mobile device capable of performing a mobile device-based life coaching method, according to some embodiments of the present invention.

FIG. 2 is a flowchart of a mobile device-based life coaching method according to some embodiments of the present invention.

FIG. 3 is a flowchart of a mobile device-based life coaching method according to other embodiments of the present invention.

FIG. 4 is a table showing examples of statistical techniques usable for a mobile device-based life coaching method, according to other embodiments of the present invention.

FIG. 5 is a table showing examples of analysis and modeling techniques and methods for checking user conditions and situations usable for a mobile device-based daily life coaching method, according to other embodiments of the present invention.

FIG. 6 is a table showing examples of analysis and modeling techniques and methods for checking user conditions and situations usable for a mobile device-based social life coaching method, according to other embodiments of the present invention.

FIG. 7 is a table showing examples of health profiling methods usable for a mobile device-based life coaching method, according to other embodiments of the present invention.

FIG. 8 is a table showing monitoring methods based on monitoring techniques usable for a mobile device-based life coaching method, according to other embodiments of the present invention.

FIG. 9 is a table showing examples of visualization methods usable for a mobile device-based life coaching method, according to other embodiments of the present invention.

FIGS. 10 and 11 are tables showing an example of coaching targets and notification, warning, or recommendation information per disease (e.g., a cold) in a mobile device-based life coaching method, according to other embodiments of the present invention.

FIG. 12 is a table showing examples of measuring sensors usable for a mobile device-based life coaching method, according to other embodiments of the present invention.

### BEST MODE

Hereinafter, the present invention will be described in detail by explaining embodiments of the invention with reference to the attached drawings.

The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thicknesses or sizes of layers are exaggerated for clarity.

The terminology used herein is for the purpose of describing particular embodiments only and is not intended to be limiting of embodiments. As used herein, the singular forms “a”, “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to schematic illustrations of idealized embodiments (and intermediate structures) of the invention. As such, variations from the shapes of the illustrations as a result, for example, of manufacturing techniques and/or tolerances, are to be expected. Thus, the embodiments of the invention should not be construed as limited to the particular shapes of regions illustrated herein, but are to include deviations in shapes that result, for example, from manufacturing.

A mobile device-based life coaching method, a mobile device, and a computer-readable recording medium having recorded the method thereon, according to some embodiments of the present invention are now described in detail with reference to the attached drawings.

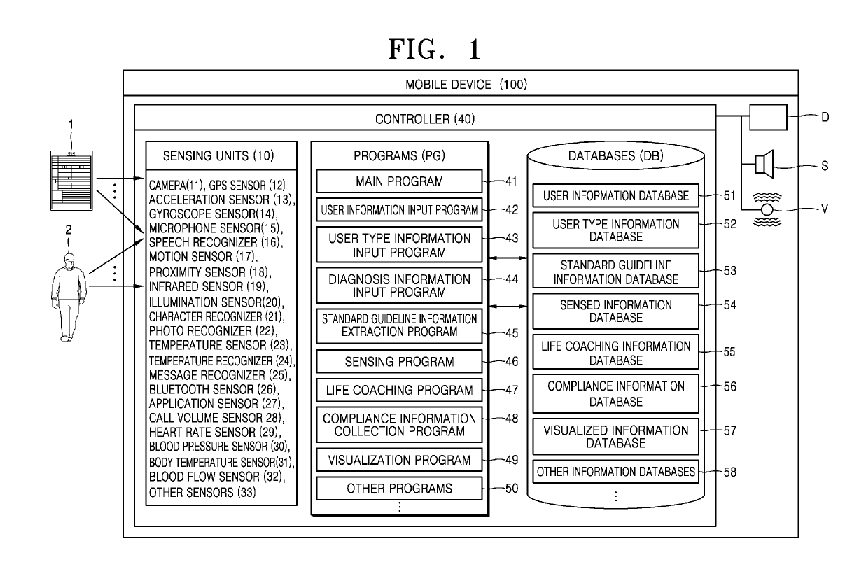

FIG. 1 is a block diagram of a mobile device 100 capable of performing a mobile device-based life coaching method, according to some embodiments of the present invention.

Initially, as illustrated in FIG. 1, the mobile device 100 according to some embodiments of the present invention may largely include a controller 40 consisting of sensing units 10, programs PG, and databases DB.

Herein, the mobile device 100 may be a smartphone capable of communication.

For example, the mobile device 100 may have a variety of applications, hybrid applications, programs, etc. installed therein, and may make phone calls to other mobile devices via a communication network. Each of the interconnected devices may not only use existing wireless communication networks including a mobile communication network such as a second generation (2G), third generation (3G), fourth generation (4G), or long term evolution (LTE) network, Wi-Fi communication network, Bluetooth communication network, cellular communication network, code division multiple access (CDMA) communication network, Ethernet communication network, WiMAX communication network, local area network (LAN), wide area network (WAN), radio frequency (RF) communication network, infrared communication network, and optical communication network, but also have an Internet browser (e.g., Netscape or Internet Explorer) capable of displaying websites in the form of HTML, XML, HTML5, or the like, an internal, external, or short-range/long-range wired/wireless network access protocol device, or the like.

However, the mobile device 100 is not limited to a smartphone and may be applied to a variety of smart devices carriable or wearable by a user 2, e.g., a smartwatch, smartband, smart clothing, personal digital assistant (PDA), smartpad, laptop computer, smart camera, smart camcorder, and electronic book.

For example, the sensing units 10 may include at least one selected from the group consisting of a camera 11, a global positioning system (GPS) sensor 12 or a Wi-Fi sensor using Wi-Fi for sensing the location of the user 2, an acceleration sensor 13 for sensing the amount of exercise or a direction, a gyroscope sensor 14, a microphone sensor 15 for sensing speech of the user 2/talks with other people/ambient noise/coughing sound/groaning sound, a speech recognizer 16, a motion sensor 17 for sensing motion, a proximity sensor 18, an infrared sensor 19, an illumination sensor 20 for sensing a sleep environment or lighting, a character recognizer 21 for sensing a stress or depression index based on search words, a photo recognizer 22 for recognizing a facial expression of the user 2, a temperature sensor 23 for sensing temperature, a temperature recognizer 24 for sensing temperature based on temperature data, a message recognizer 25 for sensing psychology of the user 2 based on messages, a Bluetooth sensor 26 for sensing other people in proximity, an application sensor 27 for sensing use of applications, a call volume sensor 28 for sensing a call volume, a weather recognizer for receiving weather information from a weather center, a heart rate sensor 29, a blood pressure sensor 30, a body temperature sensor 31, a blood flow sensor 32, other sensors 33, and combinations thereof. However, the sensing units 10 are not limited thereto and a variety of sensors or sensing circuits may be used.

For example, the controller 40 is programmed to receive user type information, extract standard guideline information corresponding to the user type information, receive sensed information from the sensing units 10, extract condition and situation information of the user 2 from the sensed information, and output life coaching information, and may be programmed by a manufacturer of the mobile device 100 or may be downloaded and installed from an online market.

For example, as illustrated in FIG. 1, the mobile device 100 may include the programs PG to be executed, and the databases DB for storing various types of information.

As illustrated in FIG. 1, the programs PG may include a main program 41 for operating all other programs, a user information input program 42 for receiving user information, a user type information input program 43 for receiving user type information, a diagnosis information input program 44 for receiving diagnosis information given by a medical expert, a standard guideline information extraction program 45 for extracting standard guideline information corresponding to the user type information, a sensing program 46 for receiving sensed information from one or more sensing units 10, a life coaching program 47 for extracting condition and situation information of the user 2 from the sensed information and outputting life coaching information corresponding to the extracted condition and situation information at an appropriate time and place in an appropriate way, a compliance information collection program 48 for collecting compliance information of the user 2, a visualization program 49 for converting the condition and situation information or the compliance information into visualized information and outputting the visualized information, and other programs 50 for supporting voice communication or data communication.

Herein, the main program 41 operates all other programs and may be a program capable of expressing output results of the other programs as screen images using a displayer D, as sound using a speaker S, or as vibration using a vibrator V in the mobile device 100, and of controlling all the other programs based on input information of the user 2.

The user information input program 42 may be a program for receiving basic information about the user 2, e.g., a name, gender, age, address, height, weight, physical strength, blood type, and social security number. The user information may be used as reference information to extract the condition and situation information or provide the life coaching information.

The user type information input program 43 is a program for receiving the user type information. The user type information may be identification information given to a group of users to which the same standard guidelines are to be applied, among all users for receiving life coaching services. For example, the user type information may be expressed as sequential codes such as “type A”, “type B”, or “type C”, or as meaningful codes such as “influenza-early stage-infant”. For instance, groups of users may be analyzed based on cluster analysis. The user type information may be extracted from the diagnosis information of the user 2, which is input using the diagnosis information input program 44, e.g., a diagnosis report 1 given by a medical expert such as a doctor, pharmacist, or nurse. A gender, age, and disease name may be disclosed in the diagnosis report 1, and the stage of disease may be inferred from a prescribed medication. Accordingly, the user type information such as “type A” or “influenza-early stage-infant” may be extracted from the diagnosis report 1.

The diagnosis information may include various types of information such as a disease name and age of the user 2, and a prescribed medication.

Accordingly, the mobile device 100 may further include the diagnosis information input program 44 for receiving the diagnosis information, and a diagnosis information database for storing the diagnosis information. The diagnosis information may include at least one selected from the group consisting of a diagnosis report photo file, text diagnosis file, diagnosis message file, email file, character recognition file, scan file, and electronic document file, which are storable in only the mobile device 100 to protect personal information, and combinations thereof. The diagnosis information input program 44 may include a character recognition program for recognizing a diagnosed disease name or a prescribed medication name in characters included in the file.

For example, the user 2 may capture an image of the diagnosis report 1 using a camera or scanner of the mobile device 100, and recognize characters in the image to automatically generate the user type information.

As described above, according to one of technical features of the present invention, all operations may be performed within the mobile device 100 as mobile device-based operations without using any network. Therefore, various types of hacking or cyberterrorism using networks may be basically disabled to ensure security, and user or patient privacy may be guaranteed as much as possible to provide reliability to users.

However, the diagnosis information of the user 2 may include information about a group to which the user 2 belongs, e.g., an office worker group, patient group, student group, or homemaker group. Herein, since users who belong to the same group may have similar conditions or situations, the same standard guideline information may be applied to the users of the same group.

Accordingly, if the information about the group to which the user 2 belongs is input, the user 2 may receive the standard guideline information corresponding thereto.

For example, the standard guideline information extraction program 45 is a program for extracting the standard guideline information corresponding to the user type information, and may be a program for extracting the standard guideline information helpful to the user 2, with reference to a variety of databases.

Herein, for example, the standard guideline information may include at least one selected from the group consisting of medication coaching information including medication time/medication compliance/over-the-counter medication information/side effects/prescription of multiple medications, hygiene coaching information including teeth brushing/hand washing/shower or bath/laundry, environment coaching information including temperature/humidity/fine dust/yellow dust/radioactivity, sanitation coaching information including mask wearing/gloves/sanitation/disinfection, exercise coaching information including exercise time/exercise intensity/the number of exercises/calorie consumption, health coaching information including mealtime/wake-up time/bedtime/habits/food tastes, and combinations thereof.

Accordingly, the standard guideline information extraction program 45 may extract various types of the standard guideline information corresponding to the user type information, and prepare to output one of the various types of the standard guideline information at an appropriate time and place in an appropriate way.

For example, the sensing program 46 is a program for receiving the sensed information from one or more sensing units 10, and may be a program capable of sequentially or simultaneously receiving the sensed information collected by the above-described various sensing units 10.

Accordingly, the mobile device 100 may sense a behavior or a health condition of the user 2 using the sensing program 46.

For example, the life coaching program 47 is a program for extracting the condition and situation information of the user 2 from the sensed information and outputting the life coaching information corresponding to the extracted condition and situation information at an appropriate time and place in an appropriate way. The sensed information collected to extract the condition and situation information may be one-off information or pattern information accumulated for a long time to show a certain pattern.

Herein, for example, the condition and situation information may include at least one selected from the group consisting of meal information for analyzing the condition and situation of a meal based on time/location/talks, going out information for analyzing the condition and situation of going out based on the location of the user 2 who leaves or returns to a residence or office, sleep information for analyzing the condition and situation of sleeping and rising based on a mobile device manipulation time or the intensity of light, exercise information for analyzing the condition and situation of exercise based on the location or acceleration of the user 2, emotion information for analyzing the condition and situation of an emotion based on a sensed talks/message/photo, eating information for analyzing the condition and situation of eating based on a sensed talks/message/location, after-work party information for analyzing the condition and situation of an after-work party based on Bluetooth/location/talks, environment information for analyzing the condition and situation of an environment based on temperature/location/time/body temperature, and combinations thereof.

The life coaching information provided based on the condition and situation information to the user at an appropriate place and time may include at least one selected from the group consisting of notification information indicating time/things to do, warning information indicating things not to do/things to be careful of, recommendation information indicating things recommended to do, and combinations thereof.

Accordingly, the mobile device 100 may infer the user type information from the diagnosis information, e.g., a disease name of the user 2 or a prescribed medication, to extract the standard guideline information helpful to the user 2, check a condition and situation of the user 2 using various sensing units, and then provide the life coaching information including at least one type of the standard guideline information, at an appropriate time and place based on the condition and situation of the user 2 in advance or later in the form of notification, warning, or recommendation information.

Therefore, the user 2 may receive the life coaching information based on his/her condition and situation at an appropriate time and place using the displayer D, the speaker S, or the vibrator V of the mobile device 100 in the form of a screen image, sound, or vibration, and easily act in compliance with the life coaching information.

Examples of life coaching may include environment coaching, medication coaching, sanitation and prophylaxis coaching, and hygiene coaching. Coaching services are provided based on certain rules. The coaching rules may specify conditions and situations which require coaching, and coaching data suitable for the conditions and situations.

For example, a coaching rule to be applied to sleep environment coaching may specify a condition of an early stage of influenza and frequent coughing, a situation in which a patient is about to go to bed at home and the humidity and temperature of a bedroom are out of humidity and temperature ranges advised by a doctor, and coaching data indicating to change a sleep environment to humidity and temperature ranges advised by a doctor or recommended in expert data. In this case, the latitude and longitude of the mobile device 100 may be determined using the GPS sensor 12 or the Wi-Fi sensor of the smartphone and, if the checked latitude and longitude are the same as or similar to those of an address of the user 2, it may be inferred that the user 2 of the mobile device 100 is at home. In addition, it may be analyzed whether a current time corresponds to a bedtime, based on a clock or the illumination sensor 20 of the smartphone.

As another example, an environment around the mobile device 100 of the user 2 may be checked using a humidity sensor, the temperature sensor 23, and the microphone sensor 15 included in the sensing units 10 of the mobile device 100. As described above, a situation of the user 2 of the mobile device 100 is comprehensively inferred and, if the situation matches a condition and situation specified by a coaching rule, coaching data specified by the coaching rule may be provided to the user 2. Alternatively, for medication coaching, it should be analyzed whether a current time corresponds to a medication time of the user 2. For sanitation and prophylaxis coaching, it should be analyzed whether the user 2 goes out. For hygiene coaching, it should be analyzed whether the user 2 is about to have or has had a meal. To analyze the situation of the user 2 as described above, the GPS sensor 12, the Wi-Fi sensor, the humidity sensor, the temperature sensor 23, the microphone sensor 15, the gyroscope sensor 14, location information, place information, etc. may be used.

For example, when the user 2 is an office worker, if a current time corresponds to a lunch hour, the location information or the place information obtained using the GPS sensor 12 or the Wi-Fi sensor indicate a restaurant, a payment confirmation message is received after a meal, and the payment confirmation message relates to the restaurant, it may be recognized that the user 2 is in the restaurant and had a meal. If the above-described information is accumulated for days, i.e., if a payment confirmation message is repeatedly received during the lunch hour, determination of a restaurant becomes more definite as time passes. If information sensed by the microphone sensor 15, e.g., ordering sound and noisy sound of a restaurant, is added, the situation of the user 2 may be more accurately inferred.

For example, mealtimes may be inferred by receiving information about scheduled breakfast, lunch, and dinner times from the user 2 at a timing when a day begins, and determining whether the user 2 has meals at the scheduled times.

Alternatively, if a payment confirmation message includes a restaurant name, it may be recognized that the user 2 has finished his/her meal, and thus a meal finished time may be determined.

If a location determined using the GPS sensor 12 or the Wi-Fi sensor does not indicate a residence or an office but indicates a restaurant, it is quite probable that the user 2 is having a meal. If a payment confirmation message includes a restaurant name, it may be recognized that the meal is finished.

If a location determined using the GPS sensor 12 or the Wi-Fi sensor does not indicate a residence or an office but indicates a restaurant, and if the Bluetooth sensor 26 senses another smartphone, which has been sensed in the office, in proximity, it is quite probable that the user 2 is having a meal together with a coworker.

If the acceleration sensor 13 and the gyroscope sensor 14 sense motion and the motion stops during a meal hour, and if the Bluetooth sensor 26 senses another smartphone, which has been sensed in the office, in proximity, it is quite probable that the user 2 is having a meal together with a coworker, for example, in a restaurant or in an office cafeteria.

If the GPS sensor 12, the acceleration sensor 13, and the gyroscope sensor 14 sense that the user 2 enters a restaurant, it may be recognized whether the user 2 has a meal, by obtaining information such as menus and servings using a speech sensor, e.g., the microphone sensor 15. For example, it may be recognized that the meal is finished, based on closing words such as “Shall we leave now?” or “Let's go.”.

For example, when the user 2 is a student, a class schedule may be received to check hours between classes. If the GPS sensor 12 or the Wi-Fi sensor sesnses that the user 2 enters a restaurant-concentrated area during a meal hour between classes, it is quite probable that the user 2 has a meal. If a payment confirmation message includes a restaurant name, it is quite probable that the meal is finished. The meal may be determined without using the GPS sensor 12 but using only the payment confirmation message received between classes. If the user 2 has the meal in a school cafeteria, the GPS sensor 12 may sense a location corresponding to a school.

Alternatively, a class schedule may be received to check hours between classes, and the acceleration sensor 13 and the gyroscope sensor 14 may sense motion during a meal hour between classes. If the motion of the smartphone stops or is greatly reduced, and if the Bluetooth sensor 26 senses another smartphone, which has been sensed in a class attended by the user 2, in proximity, it is quite probable that the user 2 is having a meal together with a classmate after the class.

It may be recognized whether the user 2 has a meal, by additionally obtaining information such as menus and servings using a speech sensor, e.g., the microphone sensor 15, and it may be recognized that the meal is finished, based on closing words such as “Let's go.” or “I'm full.”.

When the user 2 is a young woman and the GPS sensor 12 or the Wi-Fi sensor sense that the user 2 enters a restaurant (or building), if the user 2 uses a camera function during a meal hour, it is quite probable that the user 2 takes photos of food and thus it is also quite probable that the user 2 starts a meal. Considering that people move after a meal, if the acceleration sensor 13 senses a rapid acceleration, it is quite probable that the meal is finished.

When the user 2 has a meal alone at home, if the gyroscope sensor 14 senses no motion of the smartphone, it may be regarded that the smartphone is fixed. In this case, if the user 2 uses a video player function, it may be recognized that the user 2 watches a video. If a current time corresponds to a meal hour, it may be recognized that the user 2 watches a video while having a meal. Thereafter, if the video player function is paused or terminated and the smartphone moves, it may be inferred that the meal is finished.

When the user 2 has a meal alone in a restaurant since the user 2 should pay, if a payment confirmation message includes a restaurant name, it may be analyzed that a meal is finished. Alternatively, in the case of a prepaid restaurant (e.g., a fast food restaurant or a food court), it may be analyzed that a meal is started.

If the GPS sensor 12 or the Wi-Fi sensor senses that the user 2 enters a building, the acceleration sensor 13 senses no motion, and use of the smartphone is greatly increased, it may be inferred that the user uses the smartphone while having a meal.

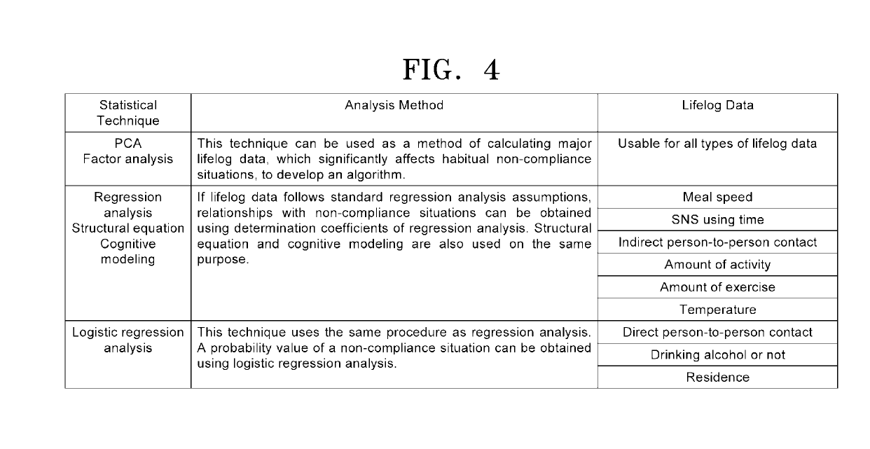

FIG. 4 is a table showing examples of statistical techniques usable for a mobile device-based life coaching method, according to other embodiments of the present invention.

As shown in FIG. 4, a variety of statistical techniques may be used to develop an algorithm for checking habitual non-compliance situations by analyzing non-compliance inducers based on locations, times, and places. In addition to the techniques shown in FIG. 4, a data mining technique and an advanced statistical technique may be used.

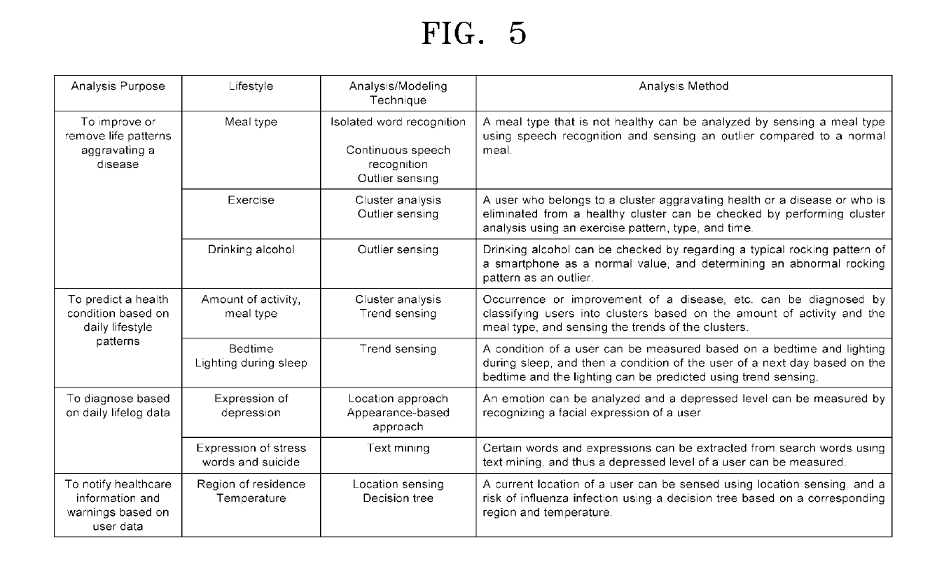

FIG. 5 is a table showing examples of analysis and modeling techniques and methods for checking user conditions and situations usable for a mobile device-based daily life coaching method, according to other embodiments of the present invention.

As shown in FIG. 5, a variety of modeling and verification methods may be used to check daily life factors related to diseases.

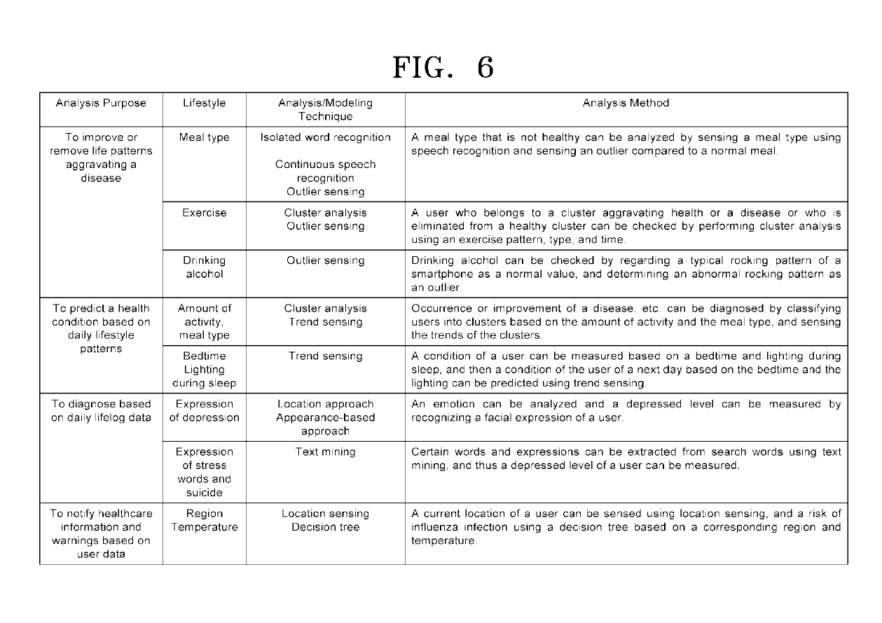

FIG. 6 is a table showing examples of analysis and modeling techniques and methods for checking user conditions and situations usable for a mobile device-based social life coaching method, according to other embodiments of the present invention.

As shown in FIG. 6, a variety of modeling and verification methods may be used to check social life factors related to diseases.

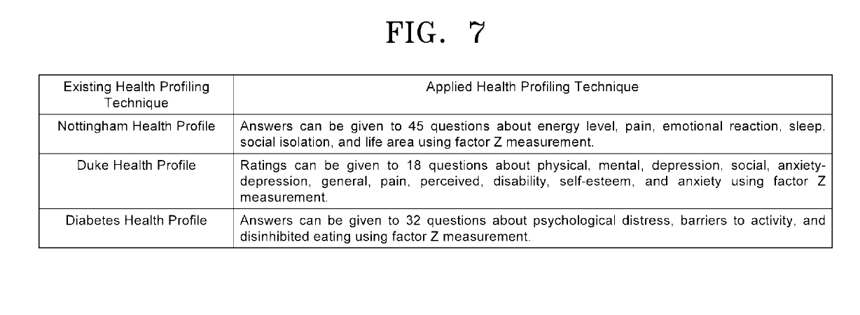

FIG. 7 is a table showing examples of health profiling methods usable for a mobile device-based life coaching method, according to other embodiments of the present invention.

As shown in FIG. 7, a personal healthcare profiling method and system based on daily and social lifelog data may be additionally implemented, profiles for user monitoring need to be set in advance, and a variety of healthcare profiling methods may be implemented using existing health profiling methods.

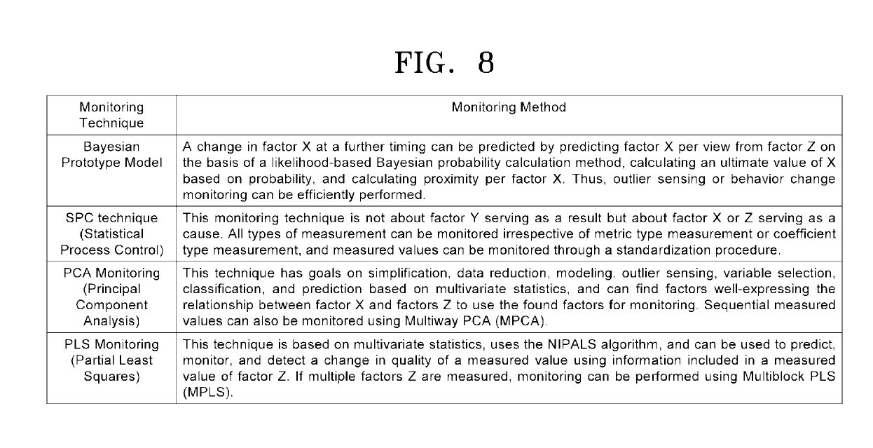

FIG. 8 is a table showing monitoring methods based on monitoring techniques usable for a mobile device-based life coaching method, according to other embodiments of the present invention.

As shown in FIG. 8, user monitoring using mobile/wearable devices in daily and social lives may be additionally implemented. Monitoring is required to predict or diagnose a health condition based on an action/behavior/activity/psychology/relationship pattern in the life of a user, and a variety of monitoring techniques may be used.

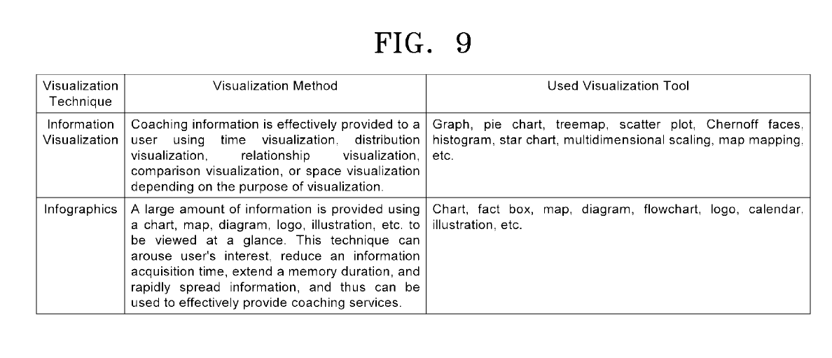

FIG. 9 is a table showing examples of visualization methods usable for a mobile device-based life coaching method, according to other embodiments of the present invention.

As shown in FIG. 9, a variety of techniques may be used to visualize coaching data, non-compliance log data, and statistics and analysis information.

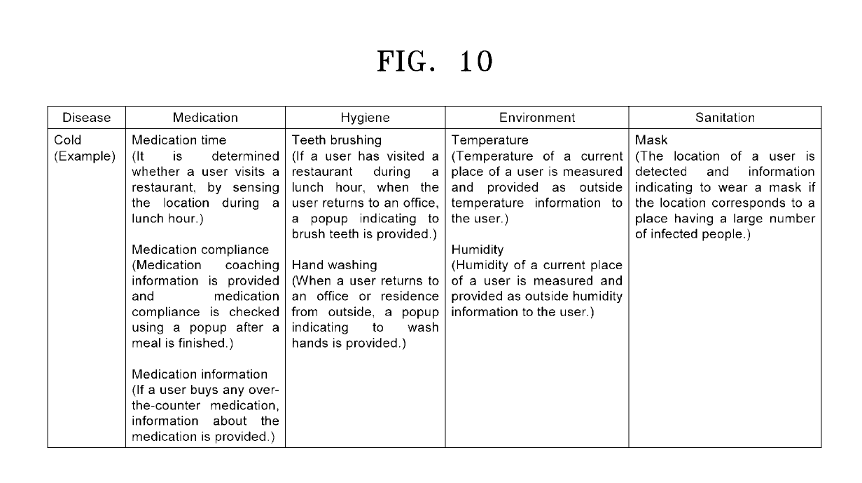

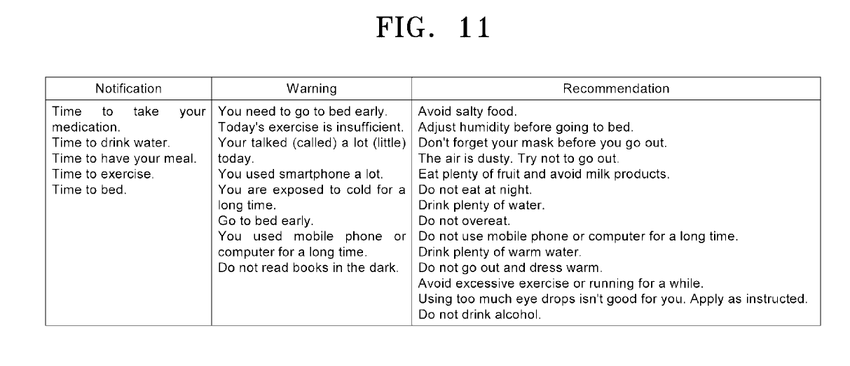

FIGS. 10 and 11 are tables showing an example of coaching targets and notification, warning, or recommendation information per disease (e.g., a cold) in a mobile device-based life coaching method, according to other embodiments of the present invention.

As shown in FIGS. 10 and 11, a life coaching information creation and transmission module may provide various types of life coaching information per disease and various types of life coaching information per combination of different types of medications. Herein, the example of FIGS. 10 and 11 relates to a cold, and the above-described life coaching information may be provided for all diseases. Herein, as shown in FIG. 11, the life coaching information to be provided may be divided into notification information, warning information, and recommendation information.

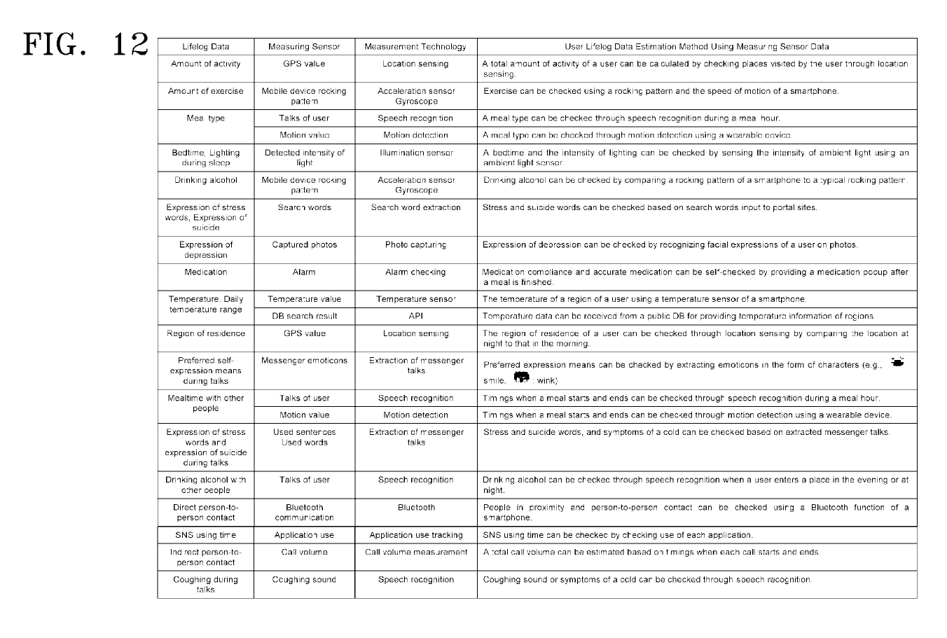

FIG. 12 is a table showing examples of measuring sensors usable for a mobile device-based life coaching method, according to other embodiments of the present invention.

As shown in FIG. 12, a basic algorithm may be created by defining lifelog data necessary for each type of life coaching and determining a technology capable of collecting the lifelog data, and an optimal algorithm may be determined through continuous prototype improvement.

For example, application user interface (UI) and back-end analysis algorithm coding may be performed using Java and Android which are computer languages required to create an Android application.

Examples of lifelog data to be used after application development, and types of smartphone sensing technologies capable of collecting the lifelog data are as shown in FIG. 12.

Meanwhile, for example, the compliance information collection program 48 is a program for collecting the compliance information of the user 2, and may be a program for collecting compliance information indicating whether the user 2 complies with the life coaching information provided from the mobile device 100. For example, the user 2 may touch a “Yes” or “No” button on a popup window asking “Did you take your medication?”.

The compliance information collection program 48 is a program for automatically collecting the compliance information of the user 2 using the sensing units 10, and may be a program for automatically collecting compliance information indicating whether the user 2 complies with the life coaching information provided from the mobile device 100.

For example, the visualization program 49 is a program for converting various types of information, e.g., the condition and situation information or the compliance information, into visualized information and outputting the visualized information. As such, the user 2 may easily check various types of information at a glance.

Herein, for example, the visualization program 49 may be inserted into an application in the form of a module by developing an R package serving as a statistical open source and capable of visualizing statistical information for statistical analysis and visualization.

Information displayed on a dashboard of a screen may be provided in multiple languages and thus Korean life coaching information given to foreign medical tourists may be translated to and provided in their languages. Accordingly, since the life coaching information may be used without interpreters, medical tours from foreign countries may be vitalized. Based on intelligibility of the user 2, the life coaching information may be provided in different levels and different expressions.

Herein, the above-described programs may be installed when manufactured by a manufacturer of the mobile device 100, or may be downloaded and installed from an online market accessible by the mobile device 100. In addition, the programs may be periodically updated.

Meanwhile, as illustrated in FIG. 1, the databases DB may include a user information database 51 for storing the user information, a user type information database 52 for storing the user type information, a standard guideline information database 53 for storing the standard guideline information, a sensed information database 54 for storing the sensed information, a life coaching information database 55 for storing the life coaching information, a compliance information database 56 for storing the compliance information, a visualized information database 57 for storing the visualized information, and other information databases 58 for storing other information.

The above-described databases DB may be updated through communication. Since the databases DB updated through communication do not include personal information, security may be ensured and personal privacy may be protected.

Herein, since the databases DB provide life coaching information appropriate for a certain prescribed medication to provide medication coaching services, relational databases may be created based on a relational database management system (RDBMS) using MySQL, Microsoft SQL Server, or the like.

Therefore, for example, according to the present invention, the mobile device 100 may include different life coaching information databases for different diseases, and a life coaching information recommendation list may be extracted when a disease name is input.

Accordingly, if a doctor gives a prescription, e.g., the diagnosis report 1, without additionally providing life coaching information, the mobile device 100, e.g., a smartphone, of a patient may read the prescription and provide life coaching information in real time. Herein, a life coaching information recommendation list may be provided based on the prescription given by the doctor. The above-described automatic recommendation may be performed by recommending a life coaching information list required for symptoms or a disease of the patient, which are inferred based on the prescription of a single or multiple medications. For example, when medications A, B, and C are prescribed, since the mobile device 100 already includes a database for storing life coaching information related to symptoms or a disease for which the medication A is prescribed, and a database for storing life coaching information related to symptoms or a disease for which the medications A and B are simultaneously prescribed, the life coaching information related to the prescription of the medication A and the life coaching information related to the prescription of the combination of the medications A and B may be extracted and provided in the recommendation list. In addition, one of a life coaching information recommendation list per disease and a life coaching information recommendation list per prescribed medication may be placed on top of the recommendation list or displayed in a pre-selected state by analyzing a previous selection history of the doctor and determining a list preferred by the doctor, thereby customizing the recommendation list.

However, the present invention is not limited to capturing of an image of the diagnosis report 1, and the diagnosis information and the life coaching information of a doctor may be transmitted directly from a doctor device to a patient device using a short-range communication network (e.g., a Bluetooth or radio-frequency identification (RFID) network). Particularly, transmission using a short-range communication network does not retain personal information in a server.

An application for recording thereon the life coaching information and the compliance information may be configured as an application for recording/storing life coaching information and compliance information based on sensing of a situation of a patient. Life coaching services may be provided by providing notification information to notify the life coaching information, providing recommendation information for efficient compliance with the life coaching information, and providing warning information when out of range.

A coaching/analysis result visualization dashboard implementable using the above-described visualization program 49 is a data dashboard for recording the life coaching information and the compliance information of the patient, and visualizing analysis results. The visualized results may be provided to the doctor on a next visit for accurate diagnosis and treatment, and may be provided to the patient to avoid undesirable situations and to be anticipatively and preventively self-cared.

The above-described dashboard may be divided into a user dashboard driven in a user device and a doctor dashboard driven in a doctor device. Unlike the user dashboard, the doctor dashboard may be driven to receive and display the life coaching information, the compliance information, and the lifelog data on a screen of the doctor device after receiving approval from the user device. In this case, a short-range communication network may be used to ensure security.

Accordingly, using the above-described programs, sensed data collected by sensing units 10 of the smartphone of the user 2, and input data collected by an input device, e.g., a keyboard, of the smartphone may be processed in the smartphone to check a condition and situation of the patient, and life coaching information stored in the smartphone may be provided if the checked condition and situation of the user 2 match a certain condition and situation of a coaching rule corresponding to the life coaching information.

That is, since all types of data collected by the mobile device 100, e.g., the smartphone, is processed in the mobile device 100 without being transmitted to a server, the mobile device 100 may check a condition and situation of the user 2 based on information extracted from the processed data, determine life coaching information based on whether the checked condition and situation match those included in the life coaching information, and output the life coaching information to the patient, thereby providing life coaching services to the patient.

As described above, the present invention is characterized in that most operations are performed in a smartphone to protect personal information, that calculations concentrated on a server is distributed to the smartphone of an individual user to easily enable real-time analysis and feedback, and that data communication between the server and the smartphone is greatly reduced. That is, if data collected by the individual user should be transmitted to a server and feedback should be received from the server, the number of communications and the amount of data communication are increased.

Furthermore, since all necessary data and algorithms are stored in the smartphone, the smartphone may independently operate even when disconnected from the server.

In addition, personal information does not need to be additionally stored in the server. For example, a Bluetooth module may be installed in a personal computer (PC) of a doctor, and then the Bluetooth module may be paired with a Bluetooth module of the smartphone of a patient to transmit a file (e.g., a prescription). Later, data indicating whether life coaching information is complied may be transmitted to the PC of the doctor through pairing. Accordingly, the personal information does not need to be stored. Alternatively, a prescription may be photographed and then read using an optical character reader (OCR). If personal information protection is not considered, the prescription may be downloaded from a server.

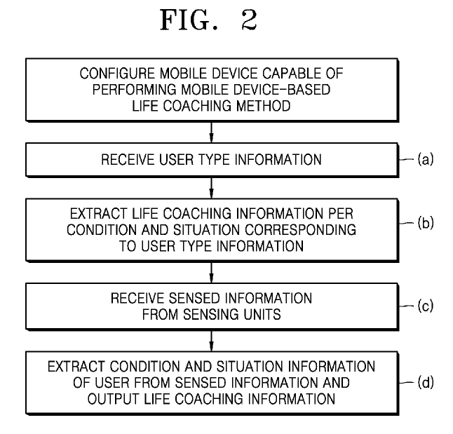

FIG. 2 is a flowchart of a mobile device-based life coaching method according to some embodiments of the present invention.

As illustrated in FIGS. 1 and 2, the mobile device-based life coaching method according to some embodiments of the present invention sequentially includes configuring the mobile device 100 capable of performing the mobile device-based life coaching method, (a) receiving user type information using the user type information input program 43, (b) extracting standard guideline information corresponding to the user type information using the standard guideline information extraction program 45, (c) receiving sensed information from one or more sensing units 10 using the sensing program 46, and (d) extracting condition and situation information of the user 2 from the sensed information and outputting life coaching information corresponding to the extracted condition and situation information at an appropriate time and place in an appropriate way using the life coaching program 47.

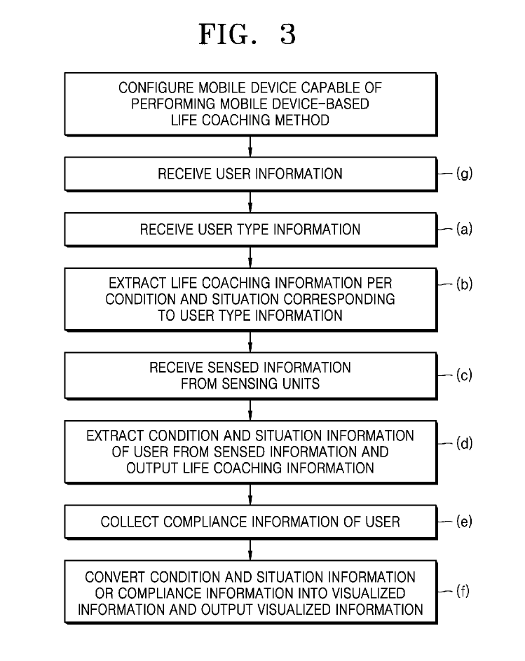

FIG. 3 is a flowchart of a mobile device-based life coaching method according to other embodiments of the present invention.

As illustrated in FIGS. 1 to 3, in the mobile device-based life coaching method according to some embodiments of the present invention, initially, (g) user information may be received on the mobile device 100 by the user information input program 42. As described above, the user information may include basic information about the user 2.

Then, (a) user type information may be received by the user type information input program 43. The user type information may be extracted from extracted from diagnosis information of the user 2, which is input using the diagnosis information input program 44, e.g., a disease name of the user 2 or prescribed medication information, which are obtainable by recognizing characters on a photo of the diagnosis report 1 captured using a camera.

Subsequently, (b) standard guideline information corresponding to the user type information may be extracted by the standard guideline information extraction program 45. For example, if the user type information indicates “influenza-early stage-infant”, the standard guideline information may include standard guideline information, e.g., sleep well, drink much water, and eat vitamin-rich foods.

Thereafter, (c) various types of sensed information may be received from one or more sensing units 10 by the sensing program 46. For example, the amount of exercise, the amount of activity, or the direction or speed of a behavior of the user 2 may be sensed using the gyroscope sensor 14 or the acceleration sensor 13, or the location of the user 2 may be sensed using the GPS sensor 12 or the Wi-Fi sensor.

Then, (d) condition and situation information of the user 2 may be extracted from the sensed information, and life coaching information corresponding to the extracted condition and situation information may be output at an appropriate time and place in an appropriate way using the life coaching program 47.

For example, if a current time corresponds to a meal hour and the user 2 moves away from an office, this may be inferred as a mealtime corresponding to a pair of condition and situation. In this case, a recommended restaurant, a recommended menu, or a message such as “Did you bring your medication?” may be output on a screen to provide various life coaching services for a cold to the user 2.

Subsequently, (e) compliance information of the user 2 may be collected by the compliance information collection program 48. For example, a message such as “Did you bring your medication?” may be output and the user 2 may input “Yes” or “No”.

Thereafter, (f) the condition and situation information or the compliance information may be converted into visualized information and the visualized information may be output by the visualization program 49. For example, various types of accumulated condition and situation information or compliance information may be provided to the user 2 in the form of various graphs, charts, or graphic images, and thus a compliance rate of the user 2 may be increased.

Herein, the present invention is not limited to the embodiments of FIGS. 2 and 3, and may be variously changed and modified without departing from the spirit and scope of the present invention. In addition, all of the above-described operations correspond to some embodiments of the present invention, are not limited to the illustrations of the drawings, and may be variously changed and modified without departing from the spirit and scope of the present invention.

The diagnosis information input in the above-described operation (a) may include information about a group to which the user 2 belongs. For example, the user 2 may select multiple groups to which he/she belongs, e.g., an office worker group, patient group, student group, or homemaker group.

For example, when the user 2 inputs diagnosis information such as male, age of 27, field worker, Internet equipment repairman, afternoon shift (13:00 to 20:00), and bad work attitude, user type information is extracted based on the above information and standard guideline information corresponding thereto may be selected and applied. If personal data is accumulated, customized guideline information may be created and complemented.

Specifically, for example, the customized guideline information is as described below.

According to the accumulated personal data of the user 2, it is analyzed that an Internet equipment repairman, B drives to repair Internet equipment on the Seoul Ring Expressway between 5:00 to 8:00 PM on Fridays in many cases, and that a service completion time in those cases is much longer than an average completion time of similar services. Based on these analysis results, guideline information such as “Do not use the Seoul Ring Expressway between 5:00 to 8:00 PM on Fridays.” may be generated and stored, and a situation (e.g., a time and place) to which the guideline information is applied may be set and stored. Thereafter, if a situation in which B goes out to repair Internet equipment between 5:00 to 8:00 PM on Friday is sensed, the guideline information such as “Do not use the Seoul Ring Expressway between 5:00 to 8:00 PM on Fridays.” may be provided to B.

In addition, various types of standard guideline information may be set for and applied to students, homemakers, etc.

Effects of the above-described present invention from various angles are as described below. Pilot tests may be performed by classifying people based on age, gender, academic background, etc. and designing clinical trials for twenty people in each group. As such, completeness of an application may be evaluated by investigating medical worker satisfaction levels and patient satisfaction levels and comparing patient lifestyle improvement rates. The application may be applied to chronic diseases, and the utility and validity thereof may be verified by doing clinical trials on depression patients. In this case, providing life coaching services at an appropriate time and place using a smartphone is much more effective compared to verbal instructions.

In addition, user-selectable coaching methods may be diversified using a machine learning function and an automatic selection function using a leaning function may be added. That is, automatic selection or setting based on situations and places is enabled.

Furthermore, a coaching UI and a statistics dashboard may be improved based on user feedback, and three coaching methods such as notification, recommendation, and warning may be upgraded to various methods (e.g., text messages, music, and vibration).

If the technical idea of the present invention is utilized in view of diagnosis, when a patient did not get better and visits a clinic or hospital again, an inappropriate part of a prescription may be checked and an improved prescription may be given to the patient.

In addition, it may be analyzed whether prescribed medications were not helpful or the patient did not take the medications in time, by checking compliance information of the patient, and thus a desirable prescription may be given based on an accurate condition of the patient.

Furthermore, characteristics in lifestyle of the patient may be checked and used for diagnosis as a cause of disease.

If the technical idea of the present invention is utilized in view of prescription, since life coaching information for a disease is provided from an already created database based on medications directly prescribed by a doctor, effective life coaching services may be provided to patients without greatly changing existing prescription systems.

Unlike a current medical system in which a doctor should directly explain life coaching information in detail to a patient, since the life coaching information is provided to a mobile device of the patient, the doctor may merely give brief information and thus consultation hours may be greatly reduced.

Furthermore, since detailed explanation of the life coaching information is provided via databases to the mobile device, the life coaching information may be provided with consistency to the patient irrespective of how detailed explanation of the life coaching information is made by the doctor.

In addition, by checking compliance information of the patient with the life coaching information to provide compliable life coaching information and to establish a treatment plan, the patient may faithfully follow instructions of the doctor.

If the technical idea of the present invention is utilized in view of life coaching, lifelog data of a mobile device may be used to determine whether a patient properly complies with life coaching information given by a doctor, and life coaching services may be continuously provided to allow the patient to behave in accordance with the life coaching information, thereby ensuring an environment capable of achieving the greatest effect of the life coaching information given by the doctor.

Furthermore, drug abuse may be avoided by providing life coaching services, and thus the burden of the doctor who diagnoses and treats a disease of the patient may be reduced. For example, when a medication time is notified, a warning about side effects of drug abuse may be provided to the patient not to take medications against the life coaching information.

In addition, by continuously providing life coaching services to the patient, the patient may be protected from misunderstanding and incorrectly complying with the life coaching information given by the doctor, and thus an unnecessary increase in a treatment period may be prevented.

Additionally, arbitrary behaviors of an elderly who has difficulty in accurately understanding instructions of the doctor may be prevented, and coaching services may be continuously provided to give appropriate information.

Meanwhile, self-care may be enabled by checking compliance with the life coaching information based on a situation (e.g., a place and time). That is, a time or place when or where the life coaching information is not complied may be checked and self-care may be enabled by avoiding the checked situation.

In terms of technology, a dominant position may be ensured in patent disputes based on a lifelog data analysis technology. That is, advantages may be achieved in patent disputes by implementing a customized service model based on a lifelog data analysis technology.

A lifelog data processing and analysis technology may be occupied in advance. By conducting research on a technology of sensing a situation of a user and providing coaching services by analyzing massive lifelog data of a mobile device, a fundamental technology of analyzing and processing massive lifelog data may be occupied.

Since new indices may be found in continuously collected personal lifelog data, lifelog data analysis may not only solve existing problems such as lack in data and absence of new indices, but also serve as a technical basis to reinterpret existing indices.

Furthermore, using new indices, the cause of a disease having no cure may be found and a cure for the disease may also be found.

Moreover, medications corresponding to life coaching information easily complied by a patient may be prescribed or developed based on the accumulated lifelog data, thereby increasing treatment effectiveness and providing customized medications.

In terms of industry, medical infrastructures and public welfare may be improved using lifelog data and mobile devices. That is, current poor medical infrastructures nearly half the OECD average may be improved using lifelog data and mobile devices and public welfare may be improved by reducing regional gaps in medical services based on a high smartphone penetration rate of Korea.

In addition, since life coaching services may be rapidly provided and popularized using lifelog data of mobile devices, ICT convergence medical industries may be vitalized.

Moreover, in a current medical system expressed as “three hour wait for three minutes with the doctor”, since a doctor should check a condition of a patient within three minutes, the quality of consultation is low and patients do not satisfy with the consultation. However, according to the present invention, since the doctor may previously check the condition of the patient based on log data, high-quality consultation may be provided within a short time, waiting time may be reduced, and satisfaction with the consultation may be increased.

To take one step forward from current medical industries focused on consultations and prescriptions of doctors, new medical services may be provided by continuously monitoring lives of patients and providing coaching information, and thus a creative economy for creating new industries based on life coaching services may be achieved.

In addition, to reduce misunderstanding due to difficult medical terminology between a doctor and a patient who use different languages, life coaching information given by the doctor may be obtained in text and provided to the patient after translating the same to a native language of the patient, thereby solving a language barrier in the medical field.

In terms of economy, unlike an existing system providing life coaching information only as verbal instructions, life coaching information and life coaching services may be received anytime anywhere using lifelog data and mobile devices, thereby popularizing and facilitating medical services.

That is, a user incapable of easily visiting a clinic or hospital may be self-cared using life coaching services, and thus exacerbation of a disease may be minimized.

As reported by the Korea Institute for Health and Social Affairs in 2011, a reduction of 20.8% in consultation time and a reduction of 4 trillion 355 billion won per year in traffic fee and opportunity cost are predicted by adopting mobile healthcare. In addition, mental and physical costs are reduced due to shortened treatment periods based on increased effects of prescriptions.

Furthermore, it is expected that life coaching services using lifelog data will enable healthy aging, a reduction in hospitalization rate (a reduction of 21%), and an effective reduction in medical expenses based on improved quality of lives (a reduction of 0.5% of GDP). That is, the quality of lives may be improved due to an economic margin based on the above-described reduction in medical expenses, thereby creating a positive cyclic loop.

In addition, the effect of creating added values by ICT-based healthcare services is predicted as about 3 trillion 80 billion won in 2020. Since the number of jobs created per turnover is large in healthcare industries, convergence between healthcare technology and ICT may rise to become a new growth engine of a country.

Moreover, by providing global medical services to increase medical convenience for foreigners and increase medical tourists, medical tourism vitalization policies promoted by government organizations and private hospitals may be supported and profits may be created by foreign capital in the medical field.

The present invention can also be embodied as computer-readable code on a computer-readable medium.

The computer-readable medium is any data storage device that can store data which can be thereafter read by a computer system.

Examples of the computer-readable medium include read-only memory (ROM), random-access memory (RAM), CD-ROMs, magnetic tapes, floppy disks, magneto-optical disks, optical data storage devices, flash memory, and carrier waves (e.g., transmission through the Internet).

The computer-readable medium can also be distributed over network coupled computer systems so that the computer-readable code is stored and executed in a distributed fashion.

While the present invention has been particularly shown and described with reference to embodiments thereof, it will be understood by one of ordinary skill in the art that various changes in form and details may be made therein without departing from the spirit and scope of the present invention as defined by the following claims.

### INDUSTRIAL APPLICABILITY

According to some embodiments of the present invention, profits may be created by selling, to users, a mobile device capable of performing a mobile device-based life coaching method by which various types of life coaching information useful to a user may be provided to the user based on a condition and situation of the user, the life coaching information may be automatically output from a mobile device, e.g., a smartphone, of a patient based on the condition and situation of the user at an appropriate time and place in an appropriate way, the life coaching information may be provided to the patient at an appropriate place and time in the form of notification/recommendation/warning information to allow the user to comply with the life coaching information, coaching data per disease and compliance information may be stored and visualized to check non-compliance and to enable active self-care, life coaching services for a disease may be continuously provided, social and economic burdens due to medical expenses may be reduced, and medical information industries may be vitalized.

1. A mobile device-based life coaching method using a mobile device carriable by a user, the mobile device comprising:
a user type information input program for receiving user type information;
a standard guideline information extraction program for extracting standard guideline information corresponding to the user type information;
a sensing program for receiving sensed information from one or more sensing units;
a life coaching program for extracting condition and situation information of the user from the sensed information and outputting life coaching information corresponding to the standard guideline information based on the extracted condition and situation information at an appropriate time and place in an appropriate way;
a user type information database for storing the user type information;
a standard guideline information database for storing the standard guideline information;
a sensed information database for storing the sensed information; and
a life coaching information database for storing the life coaching information,
the mobile device-based life coaching method comprising:
(a) receiving the user type information using the user type information input program;
(b) extracting the standard guideline information corresponding to the user type information using the standard guideline information extraction program;
(c) receiving the sensed information from the sensing units using the sensing program; and
(d) extracting the condition and situation information of the user from the sensed information and outputting the life coaching information corresponding to the standard guideline information based on the extracted condition and situation information at an appropriate time and place in an appropriate way using the life coaching program.

2. The mobile device-based life coaching method of claim 1, wherein diagnosis information from which the user type information is extractable comprises at least diagnosis information given by a medical expert and information about a group to which the user belongs,
wherein the mobile device further comprises:
a diagnosis information input program for receiving the diagnosis information; and
a diagnosis information database for storing the diagnosis information, and
wherein the mobile device comprises at least one selected from the group consisting of a smartphone, a smartwatch, a wearable smart device, and combinations thereof.

3. The mobile device-based life coaching method of claim 2, wherein the diagnosis information comprises at least one selected from the group consisting of a diagnosis report photo file, text diagnosis file, diagnosis message file, email file, character recognition file, scan file, and electronic document file, which are storable in only the mobile device to protect personal information, and combinations thereof, and
wherein the diagnosis information input program comprises a character recognition program for automatically recognizing a diagnosed disease name or a prescribed medication name in characters comprised in the file.

4. The mobile device-based life coaching method of claim 2, wherein the standard guideline information comprises at least one selected from the group consisting of:
medication coaching information comprising medication time/medication compliance/over-the-counter medication information/side effects/prescription of multiple medications;
hygiene coaching information comprising teeth brushing/hand washing/shower or bath/laundry;
environment coaching information comprising temperature/humidity/fine dust/yellow dust/radioactivity;
sanitation coaching information comprising mask wearing/gloves/sanitation/disinfection;
exercise coaching information comprising exercise time/exercise intensity/the number of exercises/calorie consumption;
health coaching information comprising mealtime/wake-up time/bedtime/habits/food tastes; and
combinations thereof.

5. The mobile device-based life coaching method of claim 2, wherein the life coaching information comprises at least one selected from the group consisting of:
notification information indicating time/things to do;
warning information indicating things not to do/things to be careful of;
recommendation information indicating things recommended to do; and
combinations thereof.

6. The mobile device-based life coaching method of claim 2, wherein the sensing units comprise at least one selected from the group consisting of a camera;
a global positioning system (GPS) sensor for sensing a location of the user;
a Wi-Fi sensor for sensing the location using Wi-Fi;
an acceleration sensor for sensing an amount of exercise, an amount of activity, or a direction;
a gyroscope sensor;
a microphone sensor for sensing speech of the user/talks with other people/ambient noise/coughing sound/groaning sound;
a speech recognizer;
a motion sensor for sensing motion;
a proximity sensor;
an infrared sensor;
an illumination sensor for sensing a sleep environment or lighting;
a character recognizer for sensing a stress or depression index based on search words;
a photo recognizer for recognizing a facial expression of the user;
a temperature sensor for sensing temperature;
a temperature recognizer for sensing temperature based on temperature data;
a message recognizer for sensing psychology of the user based on messages;
a Bluetooth sensor for sensing other people in proximity;
an application sensor for sensing use of applications;
a call volume sensor for sensing a call volume;
a weather recognizer for receiving weather information from a weather center;
a heart rate sensor;
a blood pressure sensor;
a body temperature sensor;
a blood flow sensor; and
combinations thereof.

7. The mobile device-based life coaching method of claim 2, wherein the condition and situation information comprises at least one selected from the group consisting of:
meal information for analyzing the condition and situation of a meal based on time/location/talks;
going out information for analyzing the condition and situation of going out based on the location of the user who leaves or returns to a residence or office;
sleep information for analyzing the condition and situation of sleeping and rising based on a mobile device manipulation time or intensity of light;
exercise information for analyzing the condition and situation of exercise based on the location or acceleration of the user;
emotion information for analyzing the condition and situation of an emotion based on a sensed talks/message/photo;
eating information for analyzing the condition and situation of eating based on a sensed talks/message/location;
after-work party information for analyzing the condition and situation of an after-work party based on Bluetooth/location/talks;
environment information for analyzing the condition and situation of an environment based on temperature/location/time/body temperature; and
combinations thereof.

8. The mobile device-based life coaching method of claim 1, wherein the life coaching information is output using at least a displayer, speaker, or vibrator of the mobile device in (d),
wherein the mobile device further comprises:
a compliance information collection program for collecting compliance information of the user; and
a compliance information database for storing the compliance information, and
wherein the mobile device-based life coaching method further comprises (e) collecting the compliance information of the user using the compliance information collection program, after (d).

9. The mobile device-based life coaching method of claim 1, wherein the mobile device further comprises:
a visualization program for converting the condition and situation information or the compliance information into visualized information and outputting the visualized information; and
a visualized information database for storing the visualized information, and
wherein the mobile device-based life coaching method further comprises (f) converting the condition and situation information or the compliance information into the visualized information and outputting the visualized information using the visualization program, after (d).

10. The mobile device-based life coaching method of claim 1, wherein the mobile device further comprises:
a user information input program for receiving user information; and
a user information database for storing the user information, and
wherein the mobile device-based life coaching method further comprises (g) receiving the user information using the user information input program, before (a).

11. A mobile device capable of performing a mobile device-based life coaching method, the mobile device comprising:
a user type information input program for receiving user type information;
a standard guideline information extraction program for extracting standard guideline information corresponding to the user type information;
a sensing program for receiving sensed information from one or more sensing units;
a life coaching program for extracting condition and situation information of the user from the sensed information and outputting life coaching information corresponding to the standard guideline information based on the extracted condition and situation information at an appropriate time and place in an appropriate way;
a user type information database for storing the user type information;
a standard guideline information database for storing the standard guideline information;
a sensed information database for storing the sensed information;
a life coaching information database for storing the life coaching information; and
a controller programmed to receive the user type information using the user type information input program, to extract the standard guideline information corresponding to the user type information using the standard guideline information extraction program, to receive the sensed information from the sensing units using the sensing program, and to extract the condition and situation information of the user from the sensed information and output the life coaching information corresponding to the standard guideline information based on the extracted condition and situation information at an appropriate time and place in an appropriate way using the life coaching program.

12. A computer-readable recording medium having recorded thereon a mobile device-based life coaching method using a mobile device carriable by a user, the mobile device comprising:
a user type information input program for receiving user type information;
a standard guideline information extraction program for extracting standard guideline information corresponding to the user type information;
a sensing program for receiving sensed information from one or more sensing units;
a life coaching program for extracting condition and situation information of the user from the sensed information and outputting life coaching information corresponding to the standard guideline information based on the extracted condition and situation information at an appropriate time and place in an appropriate way;
a user type information database for storing the user type information;
a standard guideline information database for storing the standard guideline information;
a sensed information database for storing the sensed information; and
a life coaching information database for storing the life coaching information, the mobile device-based life coaching method comprising:
(a) receiving the user type information using the user type information input program;
(b) extracting the standard guideline information corresponding to the user type information using the standard guideline information extraction program;
(c) receiving the sensed information from the sensing units using the sensing program; and
(d) extracting the condition and situation information of the user from the sensed information and outputting the life coaching information corresponding to the standard guideline information based on the extracted condition and situation information at an appropriate time and place in an appropriate way using the life coaching program.
